(19) United States
(12) Patent Application Publication
(10) Pub. No.: US 2009/0312693 A1
(43) Pub. Date: Dec. 17, 2009

(54) SYSTEM AND METHOD FOR DELIVERING ENERGY TO TISSUE

(75) Inventors: Hira V. Thapliyal, Los Altos, CA (US); David A. Gallup, Alameda, CA (US); James W. Arenson, Woodside, CA (US)

Correspondence Address: VytronUS, Inc. & Townsend and Townsend nd Crew LLP;Joint CN, Two Embarcadero Center, Eighth Floor, San Francisco, CA, 94111, US

(73) Assignee: VytronUS, Inc., Sunnyvale, CA (US)

(21) Appl. No.: 12/480,256

(22) Filed: Jun. 8, 2009

Related U.S. Application Data
(60) Provisional application No. 61/061,373, filed on Jun. 13, 2008.

(51) Int. Cl. A61N 7/00; A61M 37/00

(52) U.S. Cl. 604/22; 601/2

(57) ABSTRACT

Systems and methods for noninvasive skin treatment and deep tissue tightening are disclosed. An exemplary method and treatment system are configured for controlled thermal energy delivery to treat subdermal regions of the skin. First, specific control parameters such as power, skin temperature, and ultrasound frequency are chosen so as to provide localized delivery of ultrasound to a region of interest. Then, ultrasound energy is delivered at a frequency, depth, distribution, timing, and energy density to achieve the desired therapeutic effect.

### CROSS-REFERENCES TO RELATED APPLICATIONS

The present application is a non-provisional of, and claims the benefit of priority under 35 U.S.C. § 119(e) of U.S. Provisional Application No. 61/061,373 (Attorney Docket No. 027680-000300US) filed Jun. 13, 2008, the entire contents of which are incorporated herein by reference. The present application is also a non-provisional of, and claims the benefit of priority under 35 U.S.C. § 119(e) of U.S. Provisional Application No. 61/110,905 (Attorney Docket No. 027680-000800US) filed Nov. 3, 2008, the entire contents of which are incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The present invention relates generally to medical devices and methods, and more specifically to methods and systems for noninvasive skin treatment and deep tissue tightening.

Skin is the primary barrier that withstands environmental impact, such as sun, cold, wind, etc. Along with aging, environmental factors cause the skin to lose its youthful look and develop wrinkles. Human skin is made of epidermis, which is about 100 μm thick, followed by the dermis, which can extend up to 4 mm from the surface and finally the subcutaneous layer. These three layers control the overall appearance of the skin (youthful or aged). The dermis is made up of elastin, collagen, glycosoaminoglycans, and proteoglycans. The subcutaneous layer also has fibrous vertical bands that course through it and represent a link between dermal collagen and the subcutaneous layer. The collagen fibers provide the strength and elasticity to skin. With age and sun exposure, collagen loses its elasticity (tensile strength) and, as a result the skin loses its youthful, tight appearance. Not surprisingly, numerous techniques have been described for rejuvenating the appearance of skin.

One approach to skin rejuvenation is to physically inject collagen into the skin. This gives an appearance of fullness or plumpness and the offending lines are smoothened. Bovine collagen has been used for this purpose. Unfortunately, this is not a long-lasting or complete fix for the problem and there are frequent reports of allergic reactions to the collagen injections.

It is now well established that collagen is sensitive to heat treatment and denatures when heated above its transition temperature. This denaturing is accompanied by shrinking of the collagen fibers and this shrinking can provide sagging or wrinkled skin with a tightened youthful appearance. Both heat and chemical based approaches have been described and used to shrink collagen.

Peeling, or removal of, most or the entire outer layer of the skin is another known method of rejuvenating the skin. Peeling can be achieved chemically, mechanically or photothermally. Chemical peeling is carried out using chemicals such as trichloroacetic acid and phenol. An inability to control the depth of the peeling, possible pigmentary change, and risk of scarring are among the problems associated with chemical peeling.

All the above methods suffer from the problem of being invasive and involve significant amount of pain. As these cosmetic procedures are all generally elective procedures, pain and the occasional side effects have been a significant deterrent to many, who would otherwise like to undergo these procedures.

To overcome some of the issues associated with the invasive procedures, laser and radio frequency energy based wrinkle reduction treatments have been proposed. For example, U.S. Pat. No. 6,387,089 describes using pulsed light for heating and shrinking the collagen and thereby restoring the elasticity of the skin. Since collagen is located within the dermis and subcutaneous layers and not in the epidermis, lasers that target collagen must penetrate through the epidermis and through the dermis. Due to Bier's Law of absorption, the laser beam is typically the most intense at the surface of the skin. This results in unacceptable heating of the upper layers of the skin. Various approaches have been described to cool the upper layers of the skin while maintaining the layers underneath at the desired temperature. One approach is to spray a cryogen on the surface so that the surface remains cool while the underlying layers (and hence collagen) are heated. Such an approach is described in U.S. Pat. No. 6,514,244. Another approach described in U.S. Pat. No. 6,387,089 is the use of a cooled transparent substance, such as ice, gel or crystal that is in contact with the surface the skin. The transparent nature of the coolant allows the laser beam to penetrate the different skin layers.

To overcome some of the problems associated with the undesired heating of the upper layers of the skin (epidermal and dermal), U.S. Pat. No. 6,311,090 describes using RF energy and an arrangement comprising RF electrodes that rest on the surface of the skin. A reverse thermal gradient is created that apparently does not substantially affect melanocytes and other epithelial cells. However, even such non-invasive methods have the significant limitation that energy cannot be effectively focused in a specific region of interest, say, the dermis.

Other approaches have been described to heat the dermis without heating more superficial layers. These involve using electrically conductive needles that penetrate the surface of the skin into the tissue and provide heating. U.S. Pat. Nos. 6,277,116 and 6,920,883 describe such systems. Unfortunately, such an approach results in widespread heating of the subcutaneous layer and potentially melting the fat in the subcutaneous layer. This leads to undesired scarring of the tissue.

One approach that has been described to limit the general, uniform heating of the tissue is fractional treatment of the tissue, as described in U.S. Patent Publication No. 2005/0049582. This application describes the use of laser energy to create treatment zones of desired shapes in the skin, where untreated, healthy tissue lies between the regions of treated tissue. This enables the untreated tissue to participate in the healing and recovery process.

Another approach has been to thermally injure a region of tissue for treatment, as described in U.S. Patent Publication No. 2006/0122508. However, this approach relies on ultrasound to also provide imaging and monitoring of the tissue as the operator determines which regions to treat, making this approach complex and not well suited for a consumer product.

Therefore, due to the potential shortcomings of commercially available devices, it would be desirable to provide improved methods and devices that produce deep tissue tightening in a non-invasive manner. It would also be desirable if such devices delivered heat to selected target regions located at desired depths of skin, without the use of needles or other invasive methods and without reliance on ultrasound imaging.

### BRIEF SUMMARY OF THE INVENTION

The present invention generally relates to medical devices and methods and more particularly relates to devices and methods for treating tissue with ultrasound.

In a first aspect of the present invention an ultrasound based device for non-invasively treating tissue below the skin surface comprises a handpiece ergonomically shaped to fit in an operator's hand and a transducer assembly near a distal end of the handpiece. The transducer assembly is adapted to deliver ultrasound energy to the tissue. A cooling assembly is coupled with the hand piece and selectively cools the tissue surface. An electronic controller is operably connected to the ultrasound energy source. The controller and the transducer assembly are configured to treat tissue below the skin surface as the handpiece is positioned adjacent the skin surface, thereby heating a treatment zone below the skin surface without thermally damaging tissue that surrounds the treatment zone.

The transducer assembly and the cooling assembly may be integrated into a single assembly. The transducer assembly may be interchangeable with other assemblies and they may be disposable. The device may be configured to attach to a disposable unit and the disposable unit may dispense a skin care material such as a cosmeceutical, a pharmaceutical, a moisturizing agent, a skin rejuvenating agent, and combinations thereof.

The cooling assembly may cool the skin surface to about 5°-20° Celsius below ambient temperature. Cooling may be accomplished with a fluid, a gel, a jelly, or a cryogen. The cooling assembly may be adapted to maintain a skin surface temperature of about 5°-20° Celsius below ambient temperature. The cooling assembly may be housed inside the handpiece.

The transducer assembly may emit an ultrasound frequency in the range of about 1-100 MHz, or the range may be about 4-50 MHz. The cooling assembly and the transducer assembly may be configured to cause the treatment zone to be in the range of about 1-9 mm below the skin surface. The transducer assembly may be adapted to deliver energy at an angle of 65 to 115 degrees relative to the surface of the tissue.

The handpiece may comprise a plurality of apertures near a distal end thereof and the apertures may be adapted to allow a cooling fluid to pass therethrough. The apertures may be formed in a castellated pattern. The transducer assembly may be recessed from a distal end of the handpiece. Thus, while the distal end of the handpiece may contact the skin surface, the transducer assembly itself may not contact the skin. In some embodiments the transducer assembly may comprise a disc shaped transducer, and in other embodiments the transducer may have a concave or convex shaped front surface. In still other embodiments, the transducer may be annular or rectangular shaped. The transducer assembly preferably does not contact the skin surface and may be 10 mm to 15 mm away from the skin surface. The transducer assembly may comprise a plurality of transducers arranged in an array. The transducer assembly may also comprise an acoustic matching layer coupled therewith that is adapted to reduce reflection of energy from the transducer assembly back into the handpiece. The transducer assembly may also have a backing element coupled therewith that acts as a heat sink for the transducer assembly or that reflects energy from the transducer assembly distal of the handpiece. The device may also comprise a sensor that is coupled with the handpiece and adapted to detect distance between the transducer assembly and the skin surface. The handpiece may be movable relative to the skin surface and the device may comprise a motion detector adapted to detect motion of the handpiece along the skin surface, wherein the motion detector is operably coupled with the controller so that power to the transducer assembly is reduced or turned off when there is no motion.

In another aspect of the present invention, an ultrasound based device for non-invasively treating tissue below the skin surface comprises a handpiece ergonomically shaped to fit in an operator's hand and a transducer assembly near a distal end of the handpiece. The transducer assembly is adapted to deliver ultrasound energy to the tissue. A cooling assembly is coupled with the handpiece and selectively cools the tissue surface. A controller is connected to the ultrasound energy source. The controller and the transducer assembly are configured to treat tissue below the skin surface as the handpiece is positioned adjacent the skin surface without direct contact between the transducer assembly and the skin surface. This creates a heated treatment zone below the skin surface without thermally damaging tissue that surrounds the treatment zone.

In still another aspect of the present invention, a method of non-invasively treating tissue below a skin surface comprises positioning an ultrasound based treatment device adjacent the skin surface wherein the treatment device comprises a cooling assembly and a transducer assembly. The skin surface is cooled as the treatment device is disposed adjacent the skin surface. Ultrasound energy is delivered to a treatment zone below the skin surface as the treatment device is held adjacent the skin surface without direct contact between the transducer assembly and the skin surface. This results in heating the treatment zone without thermally damaging tissue surrounding the treatment zone.

The step of delivering ultrasound energy may heat collagen in the tissue thereby tightening or shrinking the collagen and minimizing the appearance of wrinkles on the surface of the skin. The ultrasound energy may also reduce fatty tissue, close varicose veins or treat cardiac tissue.

Cooling may comprise cooling the skin surface to about 5°-20° Celsius below ambient temperature. The method may further comprise maintaining a skin surface temperature of about 5°-20° Celsius below ambient temperature. The step of cooling may comprise passing a fluid past the transducer assembly, delivering a fluid to the skin surface or delivering a cooling gel, a jelly or a cryogen to the skin surface.

The step of delivering energy may comprise emitting an ultrasound frequency in the range of about 4-50 MHz and the treatment zone may be in the range of about 3-9 mm below the skin surface.

The method may further comprise adjusting an angle between the transducer assembly and the skin surface so as to control energy delivery angle. The delivery angle may be between 65 to 115 degrees relative to the surface of the tissue. The method may also comprise sensing distance between the treatment device and the skin surface. Size and depth of the treatment zone may be controlled by adjusting one of tissue surface temperature, ultrasound frequency, ultrasound energy density, velocity of the treatment device along the skin surface, and combinations thereof. The method may further comprise moving the treatment device along the skin surface. A gap of 10 mm to 15 mm between the transducer assembly and the skin surface may be maintained. Motion of the treatment device along the skin surface may also be detected. Power to the transducer assembly may be reduced or eliminated when there is no motion.

These and other embodiments are described in further detail in the following description related to the appended drawing figures.

### BRIEF DESCRIPTION OF THE DRAWINGS

FIG. 1 is a schematic illustration of an exemplary embodiment of the system.

FIG. 2 shows the distal tip assembly.

FIG. 3 illustrates the energy beam and the zone of therapy.

FIG. 4 shows another schematic illustration of an exemplary embodiment of the system.

FIGS. 5A-5C illustrate exemplary embodiments of transducer geometries.

FIGS. 5D-5F illustrate exemplary embodiments of transducer arrays.

FIG. 5G illustrates a transducer assembly and integrated cooling assembly.

FIG. 6 illustrates an ultrasound beam passing through tissue.

FIG. 7 illustrates interaction of the ultrasound beam with tissue.

FIGS. 7A-7B illustrate ablation zone shapes.

FIG. 8 illustrates the effect of surface temperature on the treatment zone.

FIG. 9 illustrates the effect of frequency on the treatment zone.

FIG. 10 illustrates the effect of energy density on the treatment zone.

FIG. 11 illustrates creation of a continuous treatment zone.

FIG. 12 illustrates creation of a variable depth continuous treatment zone.

### DETAILED DESCRIPTION OF THE INVENTION

The following description of preferred embodiments of the invention is not intended to limit the invention to these embodiments, but rather to enable any person skilled in the art to make and use this invention.

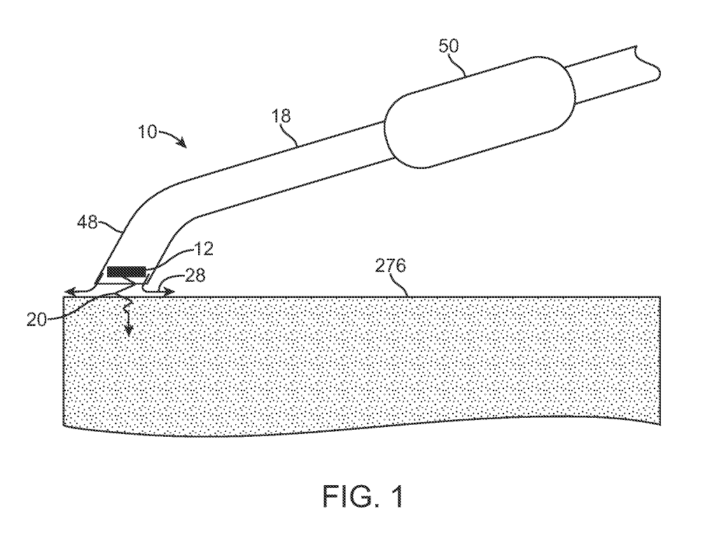

As shown in FIG. 1, the energy delivery system 10 of the preferred embodiments includes a distal tip assembly 48 to direct energy to a tissue 276. The distal tip assembly 48 includes an energy source 12 to provide a source of energy and a cooling mechanism to cool the energy source 12 and/or the tissue 276. The energy delivery system 10 is preferably designed for delivering energy to tissue, more specifically, for delivering energy to tissue that is at a depth below the outer layer(s), such as to collagen or fatty tissue located beneath the epidermis of the skin, without substantially damaging the outermost tissue layer. The energy delivery system 10, however, may be alternatively used with any suitable tissue in any suitable environment and for any suitable reason.

The Distal Tip Assembly. As shown in FIG. 1, the distal tip assembly 48 of the preferred embodiments functions to direct energy to a tissue 276 and preferably houses an energy source 12 that functions to provide a source of energy and emits an energy beam 20. The distal tip assembly 48 directs the emitted energy beam 20 from the energy source 12 to a tissue 276 and such that energy beam 20 contacts the target tissue 276 at an appropriate angle. The emitted energy beam 20 preferably contacts the target tissue at an angle between 20 and 160 degrees to the tissue, more preferably contacts the target tissue at an angle between 45 and 135 degrees to the tissue, and most preferably contacts the target tissue at an angle of 65 and 115 degrees to the tissue. The distal tip assembly 48 preferably includes a single energy source 12, but may alternatively include any suitable number of energy sources 12.

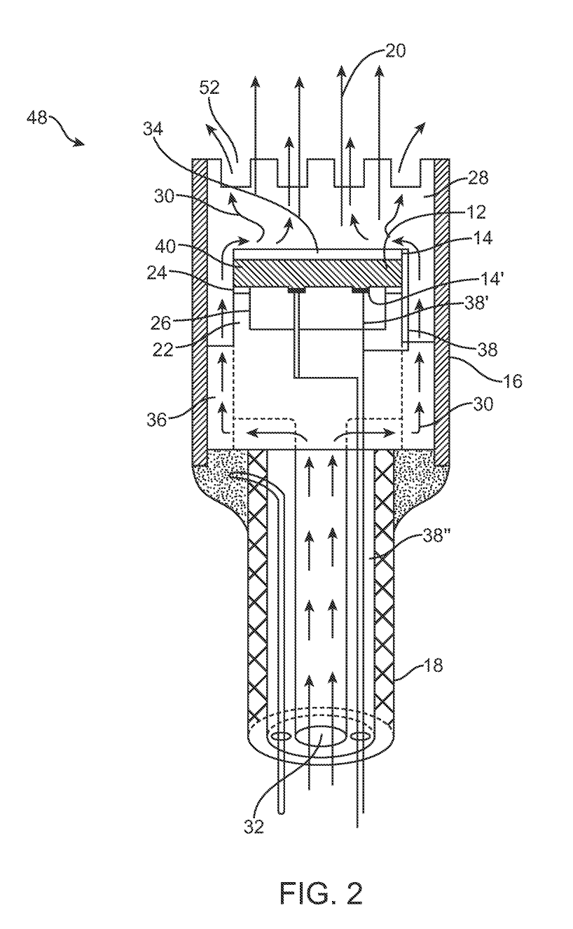

As shown in FIG. 2, the distal tip assembly 48 preferably includes a housing 16 coupled to the energy source 12. The housing is preferably an open housing 16, but may alternatively be a closed end housing that encloses the energy source 12. At least a portion of the closed end housing is made of a material that is transparent to the energy beam 20. The material is preferably transparent to ultrasound energy, such as a poly 4-methyl, 1-pentene (PMP) material or any other suitable material. The housing preferably has a rectangular or elliptical cross section, such that at least one side is longer than an adjacent side, but may alternatively have any other suitable cross section such as circular. As shown in FIG. 2, the open tubular housing preferably has a “castle head” configuration that defines a plurality of slots 52. The slots 52 function to provide exit ports for a flowing fluid or gel 28. When the front tip of the distal tip assembly 48 is in contact with or adjacent to the tissue 276 or other structures during the use of the energy delivery system 10, the slots 52 function to maintain the flow of the cooling fluid 28 past the energy source 12 and along the surface of the tissue 276. In the closed end housing, the housing defines a plurality of apertures, such as small holes towards the distal end of the housing 16. These holes provide for the exit path for the flowing fluid or gel. The apertures are preferably a grating, screen, holes, drip holes, weeping structure or any of a number of suitable apertures. The housing 16 of the distal tip assembly 48, further functions to provide a barrier between the face of the energy source 12 and the tissue 276. Because the transducer assembly is recessed in the handpiece, the distal end of the handpiece may contact the skin surface, but the transducer assembly itself preferably does not contact the skin.

The Energy Source. As shown in FIG. 1, the energy source 12 of the preferred embodiments functions to provide a source of energy and emits an energy beam 20. The energy source 12 is preferably an ultrasound transducer that emits an ultrasound beam, but may alternatively be any suitable energy source that functions to provide any suitable source of energy. Such suitable sources of energy may include radio frequency (RF) energy, microwaves, photonic energy, and thermal energy. The therapy could alternatively be achieved using cooled fluids (e.g., cryogenic fluid). The energy source and the device may be powered by an external electrical power source or they may be operated by rechargeable or non-rechargeable batteries.

The ultrasound transducer is preferably made of a piezoelectric material such as PZT (lead zirconate titanate) or PVDF (polyvinylidine difluoride), or any other suitable ultrasound beam emitting material. The transducer may further include coating layers such as a thin layer of a metal. Such suitable transducer coating metals may include gold, stainless steel, nickel-cadmium, silver, or a metal alloy. The energy source 12 is preferably one of several variations. In a first variation, as shown in FIG. 2, the energy source 12 is a disc with a flat front surface. This front surface of the energy source 12 may alternatively be either concave or convex to achieve an effect of a lens. The disc preferably has a circular geometry, but may alternatively be elliptical, polygonal, doughnut, or any other suitable shape. Additionally, different portions of the energy source 12 or different energy sources 12 may each be operated in different modes, frequencies, lengths of time, voltage, duty cycle, power, or suitable characteristic.

As shown in FIG. 2, the front face of the energy source 12 is preferably coupled to a matching layer 34. The matching layer preferably covers the front face of the energy source 12. The matching layer 34 functions to increase the efficiency of coupling of the energy beam 20 into the surrounding fluid 28. For example, when the energy source 12 is an ultrasound transducer, as the ultrasound energy moves from the energy source 12 into the fluid 28, the acoustic impedances are different in the two media, resulting in a reflection of some of the ultrasound energy back into the energy source 12. The matching layer 34 provides a path of intermediate impedance so that the sound reflection is minimized, and the output sound from the energy source 12 into the fluid 28 is maximized. The thickness of the matching layer 34 is preferably one quarter of the length of a wavelength of the sound wave in the matching layer material. The matching layer is preferably made from a plastic material such as parylene, preferably placed on the transducer face by a vapor deposition technique, but may alternatively be any suitable material, such as graphite or ceramic, added to the transducer in any suitable manner. In addition the energy source 12 may include a plurality of matching layers, generally two or three, on the face of the transducer to achieve maximum energy transmission from the energy source 12 into the fluid 28.

As shown in FIG. 2, the energy delivery system 10 of the preferred embodiments also includes a backing 22, coupled to the energy source 12. The energy source 12 is preferably bonded to the end of a backing 22 by means of an adhesive ring 24. Backing 22 is preferably made of a metal or a plastic, such that it provides a heat sink for the energy source 12. The attachment of the energy source 12 to the backing 22 is such that there is a pocket between the back surface of the energy source 12 and the backing 22. The pocket is preferably one of several variations. In a first version, the backing 22 couples to the energy source at multiple points. For example, the backing preferably includes three posts that preferably couple to the outer portion such that the majority of the energy source 12 is not touching a portion of the backing. In this variation, a fluid or gel preferably flows past the energy source 12, bathing preferably both the front and back surfaces of the energy source 12. In a second variation, the pocket is an air pocket 26 between the back surface of the energy source 12 and the backing 22. The air pocket 26 functions such that when the energy source 12 is energized by the application of electrical energy, the emitted energy beam 20 is reflected by the air pocket 26 and directed outwards from the energy source 12. The backing 22 preferably defines an air pocket of a cylindrical shape, and more preferably defines an air pocket 26 that has an annular shape. The backing defines an annular air pocket by further including a center post such that the backing has a substantially tripod shape when viewed in cross section, wherein the backing is coupled to the energy source 12 towards both the outer portion of the energy source and towards the center portion of the energy source. The air pocket 26 may alternatively be replaced by any other suitable material such that a substantial portion of the energy beam 20 is directed outwards from the energy source 12.

Cooling Mechanism. The cooling mechanism of the preferred embodiments functions to cool the energy source 12 and/or the tissue 276. The cooling mechanism functions to maintain the temperature of the energy source 12, that may become heated while being energized and emitting energy beam 20, within an optimal operating temperature range. Cooling of the energy source 12 is preferably accomplished by contacting the energy source 12 with a fluid, for example, saline or any other physiologically compatible fluid, preferably having a lower temperature relative to the temperature of the energy source 12. The temperature of the fluid or gel is preferably between −5 and 5 degrees Celsius and more preferably substantially equal to zero degrees Celsius. The fluid may alternatively be any suitable temperature to sufficiently cool the energy source 12. The cooling mechanism further functions to prevent the heating of the outer layer(s) of tissue and functions to prevent the energy delivery system 10 from substantially damaging the outer layer(s) of tissue. The cooling mechanism is preferably one of several variations.

In a first variation, as shown in FIG. 2, the cooling mechanism includes a backing 22, which preferably has a series of grooves 36 disposed longitudinally along its outer surface that function to provide for the flow of a cooling fluid 28 substantially along the outer surface of backing 22 and past the face of the energy source 12. The series of grooves may alternatively be disposed along the backing in any other suitable configuration, such as helical. The resulting fluid flow lines are depicted as 30 in FIG. 2. The flow of the cooling fluid is achieved through a lumen 32. The fluid flow lines 30 flow along the grooves in the backing 22, bathe the energy source 12, form a fluid column and exit through the slots 52 at the castle head housing 16. The fluid used for cooling the transducer preferably exits the housing 16 through the end of the housing 16 or through one or more apertures. The apertures are preferably a grating, screen, holes, drip holes, weeping structure or any of a number of suitable apertures. The fluid may alternatively flow past or bathe the energy source 12 in any other suitable fashion. The fluid 28 preferably forms a fluid column and exits the housing 16 to contact the target tissue 276 and to cool the tissue, as shown in FIG. 1.

In a second variation, the cooling mechanism includes a cooling gel or jelly. The cooling gel is preferably applied to the tissue prior to applying the energy beam 20 to the tissue. The cooling gel preferably cools the outer layer(s) of the tissue such that once the energy beam is applied to the tissue, no damage occurs to the outer layer(s). Alternatively, the cooling gel may be applied to the tissue during the use of energy delivery system 10 and preferably cools the outer layer(s) of tissue while the energy beam is applied. Furthermore, the cooling gel may additionally function to couple the energy beam 20 between the energy source 12 and patient.

In a third variation, the cooling mechanism includes a cryogen spray. The cryogen spray is preferably a cooling substance such as liquid nitrogen, but may alternatively be any other cooling spray that cools the tissue through contact cooling. The cryogen spray is preferably applied to the tissue prior to applying the energy beam 20 to the tissue. The cryogen spray preferably cools the outer layer(s) of the tissue such that once the energy beam is applied to the tissue, no damage occurs to the outer layer(s). Alternatively, the cryogen spray may be applied to the tissue during the use of energy delivery system 10 and preferably cools the outer layer(s) of tissue while the energy beam is applied.

Although the cooling mechanism is preferably one of these three variations, the cooling mechanism may be any other suitable device or substance that functions to cool the energy source 12 and/or the tissue 276.

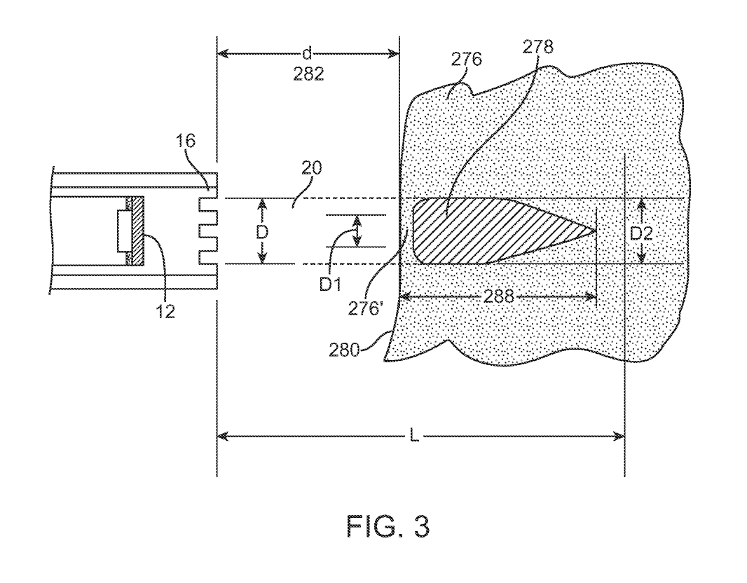

Energy Beam and Tissue Interaction. When energized with an electrical pulse or pulse train, the energy source 12 emits an energy beam 20 (such as a sound wave). The properties of the energy beam 20 are determined by the characteristics of the energy source 12, the matching layer 34, the backing 22, and the electrical pulse. These elements determine the frequency, bandwidth and amplitude of the energy beam 20 (such as a sound wave) propagated into the tissue. As shown in FIG. 3, the energy source 12 emits energy beam 20 such that it interacts with tissue 276 and forms a zone of therapy 278. For example, as described below, energy beam 20 is an ultrasound beam. The tissue 276 is preferably presented to the energy beam 20 within the collimated length L. The front surface 280 of the tissue 276 is at a distance d (282) away from the face of the housing 16. As the energy beam 20 travels through the tissue 276, its energy is absorbed by the tissue 276 and converted to thermal energy. This thermal energy heats the tissue to temperatures higher than the surrounding tissue resulting in a heated zone 278.

The energy beam 20 is preferably applied to tissue in one of several variations. The energy beam 20 is preferably applied to skin such that it interacts with the inner layers of skin below the epidermis, such as the dermis and/or the subcutaneous layer, leaving the outer layer(s) undamaged. In a first variation, the energy beam 20 interacts with the collagen located within the inner layers of the skin. During the natural aging process, exposure to UV rays, etc. collagen degenerates or breaks up, which leads to the skin becoming less firm and to the formation of wrinkles. When the energy beam 20 interacts with the collagen, it preferably heats the collagen such that the collagen tightens and/or shrinks and minimizes the appearance of wrinkles. Additionally, the heating of the collagen triggers the layers of the skin to begin their natural healing process, thereby inducing the growth of new collagen. In this variation, the depth of the energy beam 20 is preferably controlled such that the layer of fat substantially below the collagen layer preferably remains intact and/or unaffected by the energy beam 20.

In a second variation, the energy beam 20 interacts with fatty tissue located beneath the outer layers of the skin. This variation preferably functions to alter the fatty tissue to achieve clinical results substantially similar to that of conventional liposuction. In a first version, the energy beam destroys and/or liquefies the fatty tissue, removing fat cells from the patient. In a second version, the energy beam 20 functions to shrink the size of the fat chamber which may reduce the appearance of cellulite. In a third variation, the energy beam 20 interacts with and destroys the oil dispensing glands of the skin pores that lead to severe acne.

In a fourth variation, the energy beam 20 interacts with cardiac tissue. The cardiac tissue is preferably interior tissue of a chamber or a vessel of the heart, such as endocardial tissue. The energy beam 20 preferably interacts with the lower layers (such as a non-surface layer) of tissue such that the endocardial surface remains completely undamaged.

In a fifth variation, the energy beam 20 interacts with peripheral veins, preferably varicose veins. The system is positioned against the surface of the skin above the veins to be treated, but may alternatively be inserted into the vein. When the energy beam 20 interacts with the vein below the surface of the skin, the vein is heated, preferably resulting in closure of the involved vein.

Although the energy beam 20 is preferably applied to tissue in one of these variations, the energy beam may be applied to tissue in any other suitable fashion for any other suitable therapy or treatment. Other tissues that may be treated include, but are not limited to luminal tissues, and tissue where subsurface treatment is desired.

The Physical Characteristics of the Therapy Zone. The shape of the therapy zone 278 formed by the energy beam 20 depends on the characteristics of suitable combination factors such as the energy beam 20, the energy source 12 (including the material, the geometry, the portions of the energy source 12 that are energized and/or not energized, etc.), the matching layer 34, the backing 22 (described below), the electrical pulse from electrical attachments 14, 14′ (including the frequency, the voltage, the duty cycle, the length of the pulse, etc.), and the characteristics of target tissue that the beam 20 contacts and the length of contact or dwell time. Wires 38, 38′ and 38″ carry electrical energy from a power source (not illustrated) such as a battery or a wall socket to the energy source 12.

The shape of the therapy zone 278 formed by the energy beam 20 is preferably one of several variations. In a first variation, as shown in FIG. 3, the diameter D1 of the zone 278 is smaller than the diameter D of the beam 20 near the tissue surface 280 and the outer layer(s) 276′ of tissue 276 remains substantially undamaged. The change in diameters and the sparing of the outer layer(s) is due to the thermal cooling provided by the cooling mechanism that functions to cool the outer layer(s) 276′ of the tissue 276 (such as the cooling fluid 28, as shown in FIG. 1, which is flowing past the tissue surface 280). More or less of the outer layers of tissue 276′ may be spared or may remain substantially undamaged due to the amount that the tissue surface 280 is cooled and/or the characteristics of the energy source 12, the energy beam 20, etc.

As the energy beam 20 travels deeper into the tissue, the thermal cooling is provided by the surrounding tissue, which is not as efficient as that on the surface. The result is that the therapy zone 278 has a larger diameter D2 than D1 as determined by the heat transfer characteristics of the surrounding tissue as well as the continued input of the energy from the beam 20. As the beam 20 is presented to the tissue for an extended period of time, the therapy zone 278 extends into the tissue, but not indefinitely. There is a natural limit of the depth 288 of the therapy zone 278 as determined by the factors such as the attenuation of the ultrasound energy, heat transfer provided by the healthy surrounding tissue, and the divergence of the beam beyond the collimated length L. During this ultrasound-tissue interaction, the ultrasound energy is being absorbed by the tissue, and therefore less and less of it is available to travel further into the tissue. Thus a correspondingly smaller diameter heated zone is developed in the tissue, and the overall result is the formation of the heated therapy zone 278, which is in the shape of an elongated tear drop limited to a depth 288 into the tissue.

Although the shape of the therapy zone 278 is preferably one of several variations, the shape of the therapy zone 278 may be any suitable shape, at any suitable depth within the tissue, and may be altered in any suitable fashion due to any suitable combination of the energy beam 20, the energy source 12 (including the material, the geometry, etc.), the matching layer 34, the backing 22, the electrical pulse (including the frequency, the voltage, the duty cycle, the length of the pulse, etc.), the cooling mechanism, and the target tissue 276 the beam 20 contacts and the length of contact or dwell time.

Additional Elements. As shown in FIG. 1, the energy delivery system 10 of the preferred embodiments also includes an elongate member 18 coupled to the distal tip assembly 48. The elongate member 18 of the preferred embodiments is preferably a shaft having a distal tip assembly 48 and a handle 50. The elongate member 18 preferably couples the handle 50 to the distal tip assembly 48, such that the distal tip assembly 48 (and/or energy source 12) is moved along a surface of tissue 276. The shaft is preferably a flexible shaft, such that it is bent and positioned into a desired configuration. The shaft preferably remains in the desired configuration until it is re-bent or re-positioned into an alternative desired configuration. The elongate member 18 may further include a bending mechanism that functions to bend or position the elongate member 18 at various locations (such as bending a distal portion of the elongate member 18 towards the tissue 276, as shown in FIG. 1). The bending mechanism preferably includes lengths of wires, ribbons, cables, lines, fibers, filament or any other tensional member. Alternatively, the elongate member 18 may be a fixed or rigid shaft or any other suitable shaft, such as a gooseneck type shaft that includes a plurality of sections, aligned axially, that move with respect to one another to bend and position the shaft. The shaft is preferably a multi-lumen tube, but may alternatively be a catheter, a cannula, a tube or any other suitable elongate structure having one or more lumens. The elongate member 18 of the preferred embodiments functions to accommodate pull wires, fluids, gases, energy delivery structures, electrical connections, and/or any other suitable device or element.

As shown in FIG. 1, the energy delivery system 10 of the preferred embodiments also includes a handle 50 at a proximal portion of the elongate member 18. The handle 50 functions to provide a portion where an operator and/or motor drive unit couples to the system 10. The handle 50 is preferably held and moved by an operator holding the handle 50, but alternatively, the handle 50 is coupled to a motor drive unit and the movements are preferably computer controlled movements. The handle 50 may alternatively be coupled and moved in any other suitable fashion. While coupled to the handle 50 of the handheld system 10, an operator and/or motor drive unit moves the distal tip assembly 48, and/or the energy source 12, along a surface of tissue 276. The distal tip assembly 48, and the energy source 12 within it, are preferably moved and positioned within a patient such that the distal tip assembly 48 directs the emitted energy beam 20 from the energy source 12 to a tissue 276 and such that energy beam 20 contacts the target tissue 276 at an appropriate angle. The operator and/or motor drive unit preferably moves the energy delivery system 10 along a therapy path, similarly to moving a pen across a writing surface, and energizes the energy source 12 to emit energy beam 20 such that the energy source 12 provides a partial or complete zone of heating along the therapy path. The zone of heating along the therapy path preferably has any suitable geometry to provide therapy. The zone of heating along the therapy path may alternatively provide any other suitable therapy for a patient. The handle 50 may be removably coupled to a motor drive unit or may alternatively be integrated directly into the motor drive unit.

The handle 50 is preferably one of several variations. In a first variation, as shown in FIG. 1, the handle 50 is a raised portion on the elongate member 18, alternatively, the handle 50 may simply be a proximal portion of the elongate member 18 held by the operator. The handle 50 may further include finger recesses, or any other suitable ergonomic grip geometry. The handle is preferably made of a material with a high coefficient of friction, such as rubber, foam, or plastic, such that the handle 50 does not slip from the operator's hand. The handle 50 may further include controls such as dials, buttons, and an output display such that the operator may control the energy source 12, the position of the energy source 12, the cooling mechanism, the sensor (described below), the bending mechanism, and/or any other suitable element of device of the hand held system 10.

The distal tip assembly 48 of the preferred embodiments also includes a sensor that functions to detect the gap (namely, the distance of the tissue surface from the energy source 12), the thickness of the tissue 276, the characteristics of the treated tissue, the temperature at each of the various depths of tissue, and any other suitable parameter or characteristic.

The sensor is preferably an ultrasound transducer, but may alternatively be any suitable sensor to detect any suitable parameter or characteristic, such as an IR sensor, thermometer, etc. The ultrasound transducer preferably utilizes a pulse of ultrasound of short duration, which is generally not sufficient for heating of the tissue. This is a simple ultrasound imaging technique, referred to in the art as A Mode, or Amplitude Mode imaging. The sensor is preferably the same transducer as the transducer of the energy source, operating in a different mode (such as A-mode), or may alternatively be a separate ultrasound transducer. By detecting information on the gap (e.g. the distance between the transducer and the tissue surface), the thickness of the tissue targeted for therapy, the temperature at each of the various depths of tissue, and the characteristics of the heated tissue, the sensor preferably functions to guide the therapy provided by the heating of the tissue and guide the operator and/or motor drive unit as to where to position the handheld system, at what position to have the energy source with respect to the distal tip assembly in order to maintain a proper gap distance, and at what settings at which to use the energy source 12 and any other suitable elements. The gap distance is preferably between 0 mm and 20 mm, and more preferably between 10 mm and 15 mm.

Although omitted for conciseness, the preferred embodiments include every combination and permutation of the various energy sources 12, electrical attachments 14, 14′ energy beams 20, sensors 40, and processors. Additionally, other features disclosed herein may also be employed in the embodiment(s) previously described.

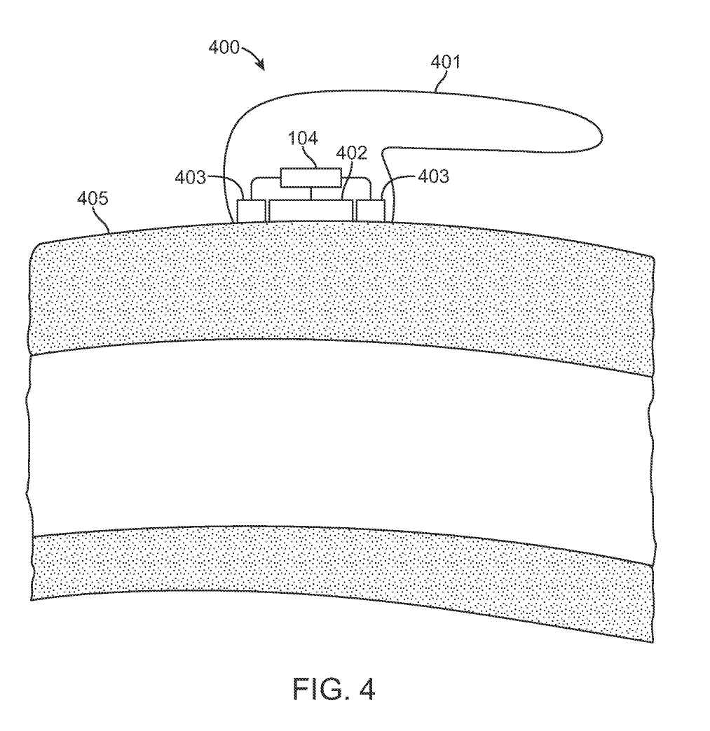

FIG. 4 illustrates another exemplary embodiment of an ultrasound based treatment device configured to treat connective tissue by providing localized thermal treatment temperatures of approximately 40° C.-90° C., and more particularly between 45° C. and 80° C., and in preferred embodiments between 50° C. and 75° C., without significant damage to surrounding and underlying skin structures, such as the subcutaneous fat layer. Following such thermal treatment, collagen fibers within targeted tissue depths shrink along their dominant direction and produce a tightening of the tissue.

The device comprises a temperature control assembly for maintaining a controlled level of temperature at the superficial tissue interface and optionally deeper into tissue. The device further comprises an ultrasound transducer assembly for delivering ultrasound energy to tissue, as well as a handpiece for allowing the user or device operator to move the device evenly along the skin surface as the cooling assembly controls the tissue surface temperature and the ultrasound assembly delivers ultrasound energy into the tissue. The device may be powered by an external power source or by an internal power source such as rechargeable or non-rechargeable batteries.

The size and depth of the treatment zones brought about by the ultrasound based thermal energy delivery within the tissue is controlled by adjusting one or more of the following parameters: tissue surface temperature, ultrasound frequency, ultrasound energy density, and the velocity with which the device is moved along the skin surface.

In accordance with an exemplary embodiment, FIG. 4 illustrates a schematic of an ultrasound based treatment device 400, configured to treat connective tissue by localized thermal treatment. Device 400 comprises a handpiece 401, an ultrasound transducer assembly 402, a cooling assembly 403, and a controller unit 404. The controller unit 404 is programmable and capable of adjusting the operating parameters of the transducer assembly 402 and cooling assembly 403. Additionally, any of the features previously described above may be used in the embodiments described hereinbelow.

The device 400 is configured to be moved along the surface of a tissue 405. As the device 400 is moved along the tissue 405, the cooling assembly 403 cools the surface of tissue 405 to a desired temperature level while the ultrasound transducer assembly 402 delivers ultrasound energy into a depth of tissue 405.

The ultrasound transducer assembly 402 comprises one or more ultrasound transducers configured for treating tissue layers and targeted regions. The transducers may optionally comprise one or more lenses in order to shape the ultrasound beams. The transducers may comprise a piezoelectrically active material, such as lead zirconate titanate (PZT), or any other piezoelectrically active material, such as a piezoelectric ceramic, crystal, plastic, and/or composite materials, as well as lithium niobate, lead titanate, barium titanate, and/or lead metaniobate. In addition to, or instead of, a piezoelectrically active material, the transducers may comprise any other materials configured for generating radiation and/or acoustical energy.

Optionally, the ultrasound transducer assembly 402 may be interchangeably attached to the device 400, for example to allow altering device parameters such as ultrasound frequency and energy density, and thereby altering the treatment by using one of a variety of interchangeable ultrasound transducer assemblies 402. Optionally, such an interchangeably attached ultrasound transducer assembly 402 may be disposable. Optionally, the device 400 may be configured to attach to a disposable unit, wherein the disposable unit dispenses skin care materials such as cosmeceuticals, pharmaceuticals, moisturizing agents, skin rejuvenating agents, and the like.

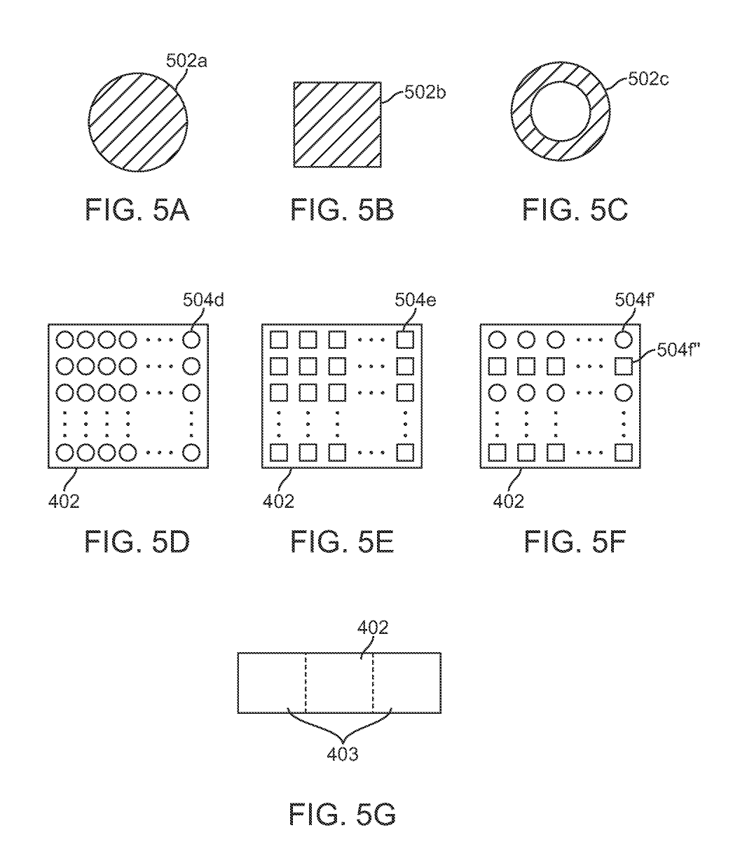

In one embodiment, transducer assembly 402 comprises a single transducer. The transducer may comprise a circular or disc-like shape 502a as shown in FIG. 5A, a rectangular or square shape 502b as shown in FIG. 5B, or a ring or annular shape 502c as shown in FIG. 5C. The shape of the transducer influences the shape of the ultrasound beam produced by the transducer, which in turn influences the shape of the treatment zone. Examples of such shapes are described further below.

Optionally, transducer assembly 402 may comprise multiple transducers arranged in an array, to deliver the ultrasound energy in such a way that the surface of the transducer assembly 402 remains cool, and/or to achieve a larger swath as the device 400 is moved. Example arrays comprising circular 504d or rectangular 504e transducers are shown in FIGS. 5D-5E, respectively. An example array comprising a mix of circular 504f and rectangular 504f′ transducers is shown in FIG. 5F. The multiple transducers may be activated separately, or together, or in varying combinations, in order to establish a desired treatment zone.

The device 400 may comprise one or more power supplies configured to provide electrical energy for the assemblies. A sense device may be provided to monitor the level of power delivered to the assemblies, including power required by one or more amplifiers or drivers in the transducer assembly 402, for safety purposes. Power sourcing components may comprise filtering configurations to increase drive efficiency and effectiveness. Alternatively, power may be applied external to device 400 through an electrical cable or other suitable means.

FIG. 4 shows device 400 containing a cooling assembly 403 inside the housing. As can be easily understood, the cooling assembly could be outside the housing as a separate unit detachably attachable to the device 400. Optionally, the cooling assembly 403 may be an integral part of the transducer assembly 402, as shown in FIG. 5G, providing cooling around the transducers and at the transducer-skin interface.

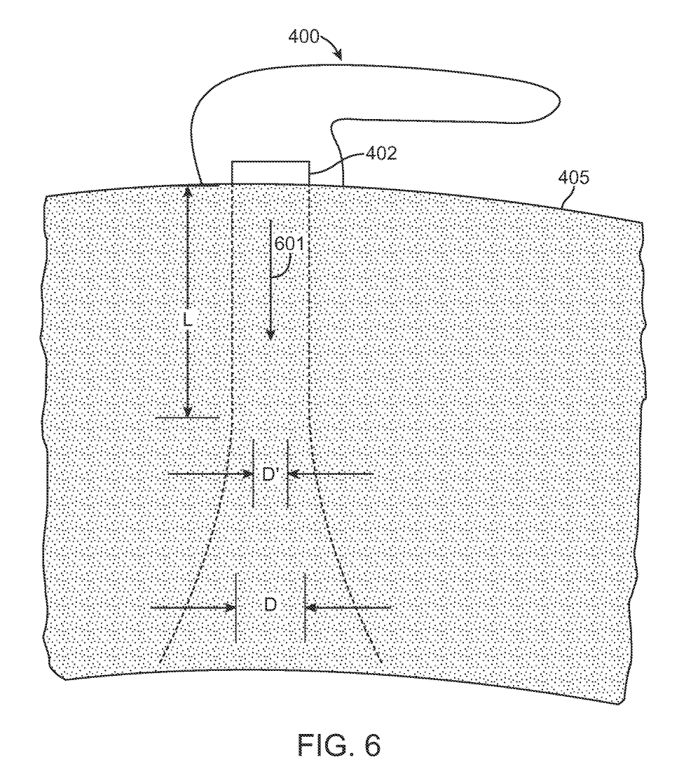

FIG. 6 shows the transducer 402 as it receives electrical energy and emits a beam 601 of ultrasound energy. A typical beam pattern is shown for the ultrasound wave as it is emitted by the transducer assembly 402, illustrating the outline of the ultrasound beam 601 by mapping where the sound pressure falls by approximately 6 decibels (dB) relative to the midline of the beam. Beam 601 travels in a generally collimated manner up to a distance of L and diverges thereafter, with the diameter at the origin of the ultrasound beam 601 corresponding approximately to the diameter D of the transducer assembly 402. If the device 400 relies on the natural focusing of a flat disc transducer, the ultrasound beam 601 converges slightly up to a depth of L, beyond which the beam diverges. The minimum beam width D′ occurs at the distance L. The distance L is determined by the diameter of the transducers (e.g., the diameter of the transducer disc) and the ultrasound frequency. Further details on the behavior of the beam 601 and configuring the transducer assembly 402 (such as using various types transducers or transducer arrays, using acoustic lenses, etc.) are described in co-pending U.S. Patent Publication No. 2007/0265609 having common inventors and assignee of the present application.

Still referring to FIG. 6, for device 400, a relatively large L is desired as it establishes the size or volume of the treatment zone, and therefore D is maximized for a given device diameter so that L is in turn maximized. Since a higher ultrasound frequency increases the distance L, and ultrasound is attenuated in the tissue 105 more with increasing ultrasound frequency, the desired depth of the treatment zone determines the useable maximum frequency of the ultrasound. Given the constraints of device size and ultrasound attenuation, the present device may use, for example, an operating frequency of about 12 MHz and a disc diameter of about 2.5 mm, resulting in a depth L of about 12 mm and a minimum beam width D′ of about 1.6 mm.

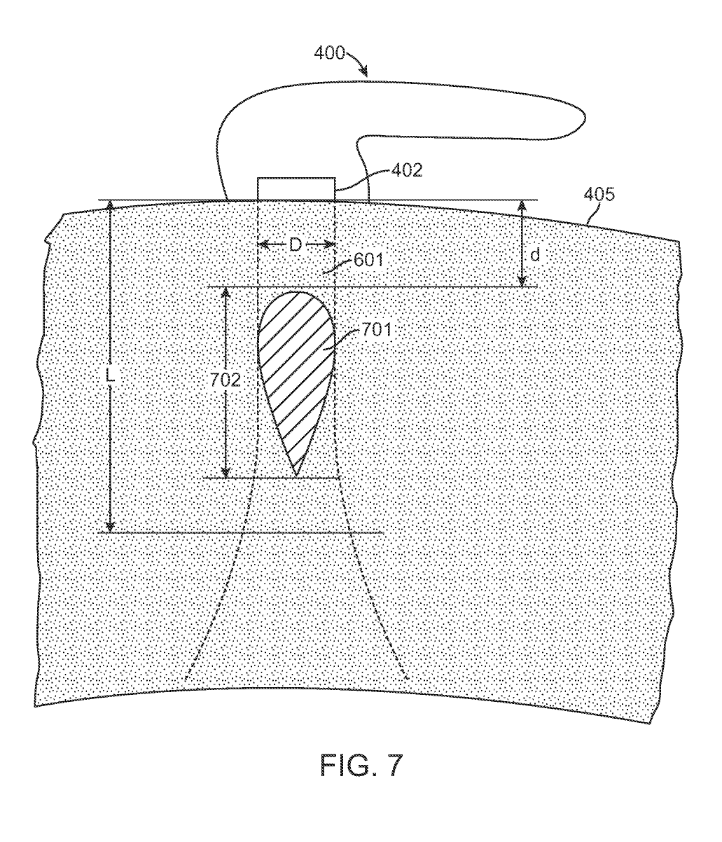

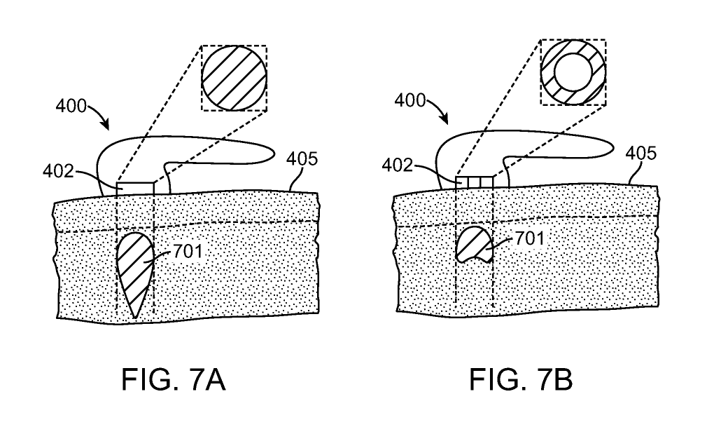

FIG. 7 shows the interaction of the ultrasound beam with the tissue. The tissue 405 is presented to the ultrasound beam 601 within the collimated length L. As the ultrasound beam 601 travels through the tissue 405, its energy is absorbed by the tissue 405 and converted to thermal energy which heats the tissue to temperatures higher than the surrounding tissue. The result is a heated treatment zone 701 of length 702 which has a typical shape of an elongated tear drop, starting at a distance d away from the face of the device 400 and below the surface of the tissue 405. Further details on the tissue heat transfer characteristics shaping the heated treatment zone 701 are described in the above referenced U.S. Patent Publication No. 2007/0265609. As described above, the shape of the transducer influences the shape of the treatment zone, and FIGS. 7A-7B illustrate two examples of this. FIG. 7A shows an elongated tear-drop shaped treatment zone 701 as produced by a disk-shaped transducer, while FIG. 7B shows a less elongated tooth-shaped treatment zone 701 as produced by a ring-shaped transducer. Other transducer shapes may produce yet differently shaped treatment zones. Using different transducer shapes allows an operator to shape the treatment zone appropriately and thereby to spare selective portions of tissue, such as the fat layer or nerve tissue, from thermal injury.

As mentioned above, the delivery of ultrasound energy at a suitable depth, distribution, timing, and energy density is provided by adjusting the parameters of device 400 in order to achieve the desired therapeutic effect of localized thermal energy delivery to tissue 405. Thus, the parameters of the device 400 may be advantageously adjusted to target a particular region of interest within tissue 405, for example as defined by such a target region's depth and shape. Such a target region may substantially reside entirely within a specific layer of the tissue, such as within the fascia, or it may cross a combination of tissue layers such as skin, dermis, fat/adipose tissue, fascia, suspensory tissue, or muscle. We now turn to describing the various parameters of the device 400 in further detail.

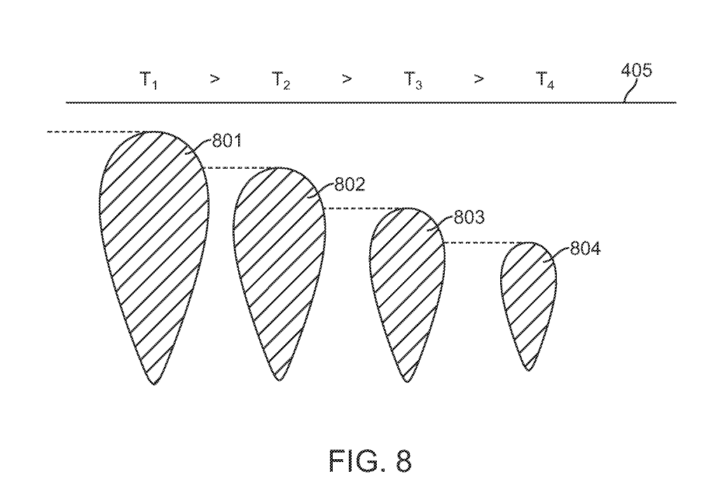

Tissue Surface Temperature: One parameter of the ultrasound based treatment device 100 is the local tissue surface temperature. In general, lowering the local tissue surface temperature tends to cause the treatment zones to be created at a larger depth below the tissue surface, while conversely increasing the temperature tends to cause the treatment zones to be created at a smaller depth. This is shown diagrammatically in FIG. 8, wherein a series of decreasing surface temperatures T1>T2>T3>T4 result in decreasing treatment zones 801, 802, 803 and 804. Thus, one way to adjust the superficial treatment depth is by modifying the local tissue surface temperature as controlled and maintained by the cooling assembly 403.

In one embodiment, the cooling assembly 403 comprises a highly conductive material, such as a metal plate, which transfers heat away from the tissue 405, thereby cooling the tissue. In another embodiment, the cooling assembly 403 is configured to spray a coolant onto the surface of the tissue 405, thereby cooling the tissue 405. In another embodiment, the cooling assembly 403 uses the flow of a chilled fluid, or a gel or similar substance that absorbs heat from its surroundings and as a result undergoes a phase transition, in order to remove heat from the tissue 405. In yet another embodiment, the cooling assembly 403 comprises a Peltier cooling device or a Thomson cooling device for selectively cooling tissue 405. In another embodiment, the cooling assembly 403 uses a gel or fluid as a thermal coupler to increase the flow of heat from the tissue 405 into the cooling assembly 403.

In one embodiment, the cooling assembly 403 is configured to maintain a local tissue surface temperature of about 5° C.-10° C. below the ambient temperature while the transducer assembly 402 delivers ultrasound energy into tissue 405. The cooling assembly 403 preferably monitors the temperature profile of the local tissue surface and suitably adjusts the cooling level to maintain the desired temperature.

In one embodiment, the cooling assembly 403 is configured to reduce the surface temperature of the surface of the transducer assembly 402, thereby assisting in cooling the surface of tissue 405.

Ultrasound Frequency: A second parameter of the ultrasound based treatment device 400 is the frequency of the ultrasound beam. In general, increasing the ultrasound frequency causes the ultrasound energy to be absorbed more quickly in the tissue 405 and to dissipate closer to the surface of tissue 405, whereas decreasing the ultrasound frequency causes the ultrasound energy to penetrate further into tissue 405 and dissipate at a larger depth.

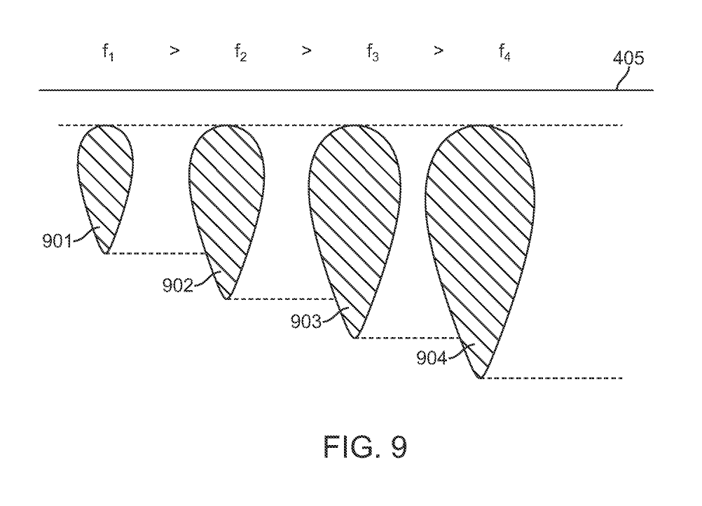

This is shown diagrammatically in FIG. 9, wherein a series of decreasing ultrasound frequencies f1>f2>f3>f4 result in increasing treatment depths 901, 902, 903 and 904. Thus, modifying the ultrasound frequency generated by the transducer assembly 402 represents another way of adjusting the treatment depth and size of treatment zones and thereby the location of the treatment zone. In one embodiment, the transducer assembly 402 is configured to deliver ultrasound energy at a frequency in the range of approximately 1-400 MHz, and typically between 1-100 MHz, for therapy applications.

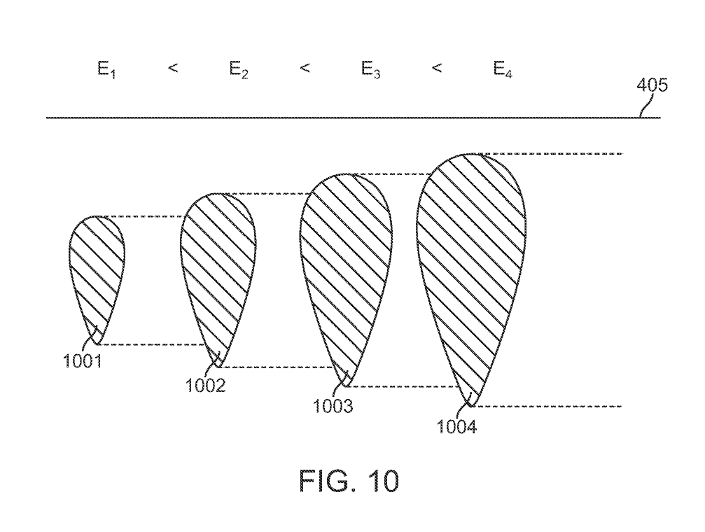

Ultrasound Energy Density: A third parameter of the ultrasound based treatment device 400 is the ultrasound energy density as delivered by the ultrasound beam. The ultrasound energy density determines the speed at which the treatment occurs. The acoustic power delivered by the transducer assembly 402 divided by the cross sectional area of the beam width determines the power density or the energy density per unit time. Increasing the ultrasound energy density results in larger amounts of heat delivered to the tissue per unit time and therefore in larger treatment zone sizes, while decreasing the ultrasound energy density results smaller treatment zone sizes. This is shown diagrammatically in FIG. 10, wherein several treatment zones are created in the tissue 405 but with varying levels of ultrasound energy density, illustrating that increasing energy density levels E1<E2<E3<E4 result in increasing treatment zone sizes 1001, 1002, 1003 and 1004. In this invention, effective acoustic power ranges from 0.3 Watts to >10 Watts, and the corresponding power densities range from 3 Watts/cm² to >100 Watts/cm². These power densities are developed in the treatment zone. As the beam diverges beyond the treatment zone, the power density falls such that treatment will not occur, regardless of the time exposure. In one embodiment, with sufficient power density, 1-10 seconds of treatment time delivers sufficient energy density to develop a treatment zone.

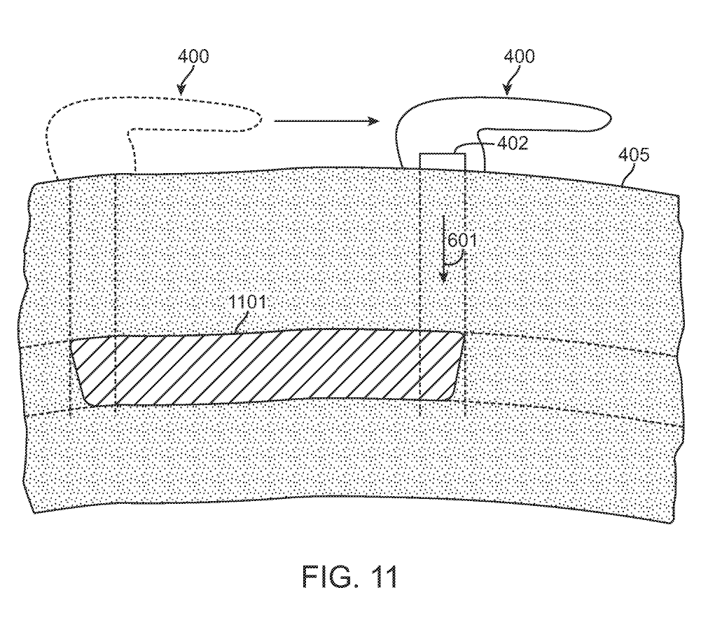

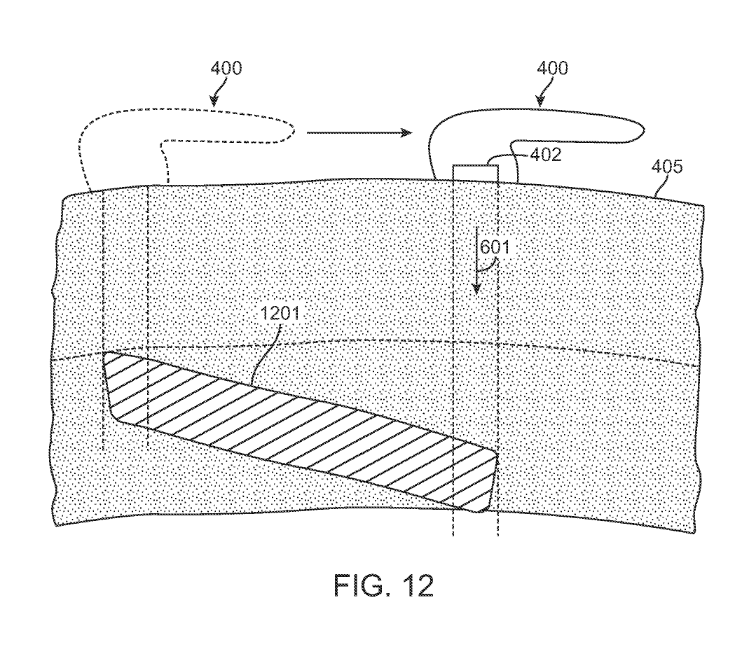

Motion of Device Along the Skin Surface: A fourth parameter of the ultrasound treatment device 400 is the speed with which an operator moves the device 400 along the surface of the skin 405, as shown in FIG. 11. Generally, the device 400 should be moved at a rate that is slow enough to allow the ultrasound beam 601 to sufficiently heat a target region to provide treatment. At the same time, the device 400 should be moved across the tissue at a predetermined rate in order to complete the treatment procedure in a practical time limit. As a result, as the operator moves the device 400 along the surface of the skin 405 in a controlled manner and at a controlled speed, a continuous treatment zone is created at the chosen depth below the skin surface. This is shown in FIG. 11, wherein the indicated motion of device 400 causes the creation of a continuous treatment zone 1101. Note that while FIG. 11 shows the treatment zone 1101 extending substantially parallel to the surface of tissue 405, it is possible to produce a treatment zone 1101 that extends across varying depths within tissue 405. This can be achieved by a corresponding modification of one or more parameters of device 400 during treatment and as device 400 moves along the surface of tissue 405. For example, to generate a treatment zone 1201, which is at an angle to the surface of tissue 405, as shown in FIG. 12, the surface could be cooled at progressively higher rates in the direction of the device movement. It is noted that the device 400 may be moved in a linear fashion along the skin, or it may be moved in a non-linear fashion, such as in a circular or zig-zag fashion, in order to produce desired treatment zones. Furthermore, as an alternative to manually moving the device 400 along the skin, the movement of the device 400 may be motorized.

Optionally, the device 400 may be configured to operate such that it prevents or inhibits excessive heat delivery to a treatment zone, thereby providing increased safety. In one such embodiment, device 400 limits the ultrasound power density to a level that does not excessively heat a treatment zone, even when device 400 remains stationary on the tissue surface for an extended period of time. In one embodiment, such an upper limit on the power density is set to about 10-100 Watts/cm², preferably to about 20-60 Watts/cm², and more preferably to about 30-50 Watts/cm².

In another such embodiment for preventing or inhibiting excessive heat delivery to a treatment zone within tissue 405, device 400 is configured to sense motion of the device 400 relative to tissue surface. When the device 400 determines it is not moving with sufficient speed along the surface of tissue 405, it reduces or shuts off ultrasound energy delivery. In order to detect motion of the device 400 relative to the tissue surface, the device 400 may comprise various motion and/or position sensors, such as accelerometers, encoders or other position/orientation devices). In one embodiment, a computer mouse is used to detect the motion, while a computer controls the power delivery to the device 400.

By adjusting the above parameters, spatial control of treatment depth may be suitably adjusted in various ranges, such as within a wide range of approximately 0 to 15 mm of depth, suitably fixed to a few discrete depths for typical usage, with an adjustment limited to a fine range, for example approximately between 0 to 9 mm. Alternatively or in combination, one or more parameters of device 400 may be dynamically adjusted during treatment.

While the above is a complete description of the preferred embodiments of the invention, various alternatives, modifications, and equivalents may be used. For example, features described herein my be interchanged with one another as desired. Therefore, the above description should not be taken as limiting the scope of the invention which is defined by the appended claims.

What is claimed is:

1. An ultrasound based device for non-invasively treating tissue below the skin surface, said device comprising:
a handpiece ergonomically shaped to fit in an operator's hand;
a transducer assembly near a distal end of the handpiece, the transducer assembly adapted to deliver ultrasound energy to the tissue;
a cooling assembly for selectively cooling the tissue surface, the cooling assembly coupled with the handpiece; and
a controller operably connected to the ultrasound energy source,
wherein the controller and the transducer assembly are configured to treat tissue below the skin surface as the handpiece is positioned adjacent the skin surface, thereby heating a treatment zone below the skin surface without thermally damaging tissue that surrounds the treatment zone.

2. The device of claim 1, wherein the transducer assembly and the cooling assembly are integrated into a single assembly.

3. The device of claim 1, wherein the transducer assembly is interchangeable.

4. The device of claim 3, wherein the transducer assembly is disposable.

5. The device of claim 1, wherein the device is configured to attach to a disposable unit, wherein the disposable unit dispenses a skin care material.

6. The device of claim 5, wherein the skin care material comprises one of a cosmeceutical, a pharmaceutical, a moisturizing agent, a skin rejuvenating agent, and combinations thereof.

7. The device of claim 1, wherein the cooling assembly cools the skin surface to about 5°-20° Celsius below ambient temperature.

8. The device of claim 7, wherein the cooling assembly cooling the skin with a fluid, a gel, a jelly, or a cryogen.

9. The device of claim 7, wherein the cooling assembly maintains a skin surface temperature of about 5°-20° Celsius below ambient temperature.

10. The device of claim 1, wherein the cooling assembly is housed inside the handpiece.

11. The device of claim 1, wherein the transducer assembly emits an ultrasound frequency in the range of about 1-100 MHz.

12. The device of claim 11, wherein the frequency range is about 4-50 MHz

13. The device of claim 1, wherein the cooling assembly and the transducer assembly are configured to cause the treatment zone to be in the range of about 1-9 mm below the skin surface.

14. The device of claim 1, wherein the transducer assembly is adapted to deliver energy at an angle of 65 to 115 degrees relative to the surface of the tissue.

15. The device of claim 1, wherein the handpiece comprises a plurality of apertures near a distal end thereof, the apertures adapted to allow a cooling fluid to pass therethrough.

16. The device of claim 15, wherein the apertures are formed in a castellated pattern.

17. The device of claim 1, wherein the transducer assembly is recessed from a distal end of the handpiece.

18. The device of claim 17, wherein the transducer assembly does not contact the skin surface.

19. The device of claim 18, wherein the transducer assembly is disposed 10 mm to 15 mm away from the skin surface.

20. The device of claim 1, wherein the transducer assembly comprises disc shaped transducer.

21. The device of claim 1, wherein the transducer assembly comprises a transducer having a concave or convex shaped front surface.

22. The device of claim 1, wherein the transducer assembly comprises an annular or rectangular shaped transducer.

23. The device of claim 1, wherein the transducer assembly comprises a plurality of transducers arranged in an array.

24. The device of claim 1, wherein the transducer assembly comprises a matching layer coupled therewith, the matching layer adapted to reduce reflection of energy from the transducer assembly back into the handpiece.

25. The device of claim 1, wherein the transducer assembly comprises a backing element coupled therewith, the backing element acting as a heat sink for the transducer assembly.

26. The device of claim 1, wherein the transducer assembly comprises a backing element coupled therewith, the backing element adapted to reflect energy from the transducer assembly distal of the handpiece.

27. The device of claim 1, further comprising a sensor coupled with the handpiece and adapted to detect distance between the transducer assembly and the skin surface.

28. The device of claim 1, wherein the handpiece is movable relative to the skin surface and the device further comprises a motion detector adapted to detect motion of the handpiece along the skin surface, wherein the motion detector is operably coupled with the controller so that power to the transducer assembly is reduced or turned off when there is no motion.

29. An ultrasound based device for non-invasively treating tissue below the skin surface, said device comprising:
a handpiece ergonomically shaped to fit in an operator's hand;
a transducer assembly near a distal end of the handpiece, the transducer assembly adapted to deliver ultrasound energy to the tissue;
a cooling assembly for selectively cooling the tissue surface, the cooling assembly coupled with the handpiece; and
a controller operably connected to the ultrasound energy source,
wherein the controller and the transducer assembly are configured to treat tissue below the skin surface as the handpiece is positioned adjacent the skin surface without direct contact between the transducer assembly and the skin surface, thereby heating a treatment zone below the skin surface without thermally damaging tissue that surrounds the treatment zone.

30. The device of claim 29, wherein the transducer assembly is recessed from a distal end of the handpiece.

31. The device of claim 29, wherein the transducer assembly emits an ultrasound frequency in the range of about 1 to 100 MHz.

32. The device of claim 29, wherein the handpiece comprises a plurality of apertures near a distal end thereof, the apertures adapted to allow a cooling fluid to pass therethrough.

33. The device of claim 29, wherein the transducer assembly is disposed 10 mm to 15 mm away from the skin surface.

34. A method of non-invasively treating tissue below a skin surface, said method comprising:
positioning an ultrasound based treatment device adjacent the skin surface, the treatment device comprising a cooling assembly and a transducer assembly;
cooling the skin surface as the treatment device is disposed adjacent the skin surface; and
delivering ultrasound energy to a treatment zone below the skin without direct contact between the transducer assembly and the skin surface, thereby heating the treatment zone without thermally damaging tissue surrounding the treatment zone.

35. The method of claim 34, wherein the step of delivering ultrasound energy heats collagen in the tissue thereby tightening or shrinking the collagen and minimizing the appears of wrinkles on the surface of the skin.

36. The method of claim 34, wherein the step of delivering ultrasound energy reduces fatty tissue.

37. The method of claim 34, wherein the step of delivering ultrasound energy closes varicose veins.

38. The method of claim 34, wherein the tissue comprises cardiac tissue.

39. The method of claim 34, wherein the step of cooling comprises cooling the skin surface to about 5°-20° Celsius below ambient temperature.

40. The method of claim 39, further comprising maintaining a skin surface temperature of about 5°-20° Celsius below ambient temperature.

41. The method of claim 34, wherein the step of cooling comprises passing a fluid past the transducer assembly.

42. The method of claim 34, wherein the step of cooling comprises delivering a fluid to the skin surface.

43. The method of claim 34, wherein the step of cooling comprises delivering a cooling gel, a jelly or a cryogen to the skin surface.

44. The method of claim 34, wherein the step of delivering comprises emitting an ultrasound frequency in the range of about 4-50 MHz.

45. The method of claim 34, wherein the treatment zone is in the range of about 1-9 mm below the skin surface.

46. The method of claim 34, further comprising adjusting an angle between the transducer assembly and the skin surface so as to control energy delivery angle.

47. The method of claim 46, wherein the energy delivery angle is between 65 to 115 degrees relative to the surface of the tissue.

48. The method of claim 34, further comprising sensing distance between the treatment device and the skin surface.

49. The method of claim 34, further comprising controlling size and depth of the treatment zone by adjusting one of tissue surface temperature, ultrasound frequency, ultrasound energy density, velocity of the treatment device along the skin surface, and combinations thereof.

50. The method of claim 34, further comprising moving the treatment device along the skin surface.

51. The method of claim 50, wherein the step of moving the treatment device comprises maintaining a gap of 10 to 15 mm between the transducer assembly and the skin surface.

52. The method of claim 50, further comprising detecting motion of the treatment device along the skin surface and reducing or eliminating power to the transducer assembly when there is no motion.
